(19) United States
(12) Patent Application Publication
(10) Pub. No.: US 2022/0218282 A1
(43) Pub. Date: Jul. 14, 2022

(54) SYSTEMS AND METHODS FOR ALIGNMENT OF SENSORS ACROSS A CHEST OF A TARGET PATIENT

(71) Applicant: Sensible Medical Innovations Ltd., Netanya (IL)

(72) Inventors: Amir SAROKA, Herzlia (IL); Anat SHANI, Tel-Aviv (IL); Shlomi BERGIDA, Ein Sarid (IL); Liran MISHANI, RaAnana (IL); Naama BERGMAN, Tel-Aviv (IL); Arin MAZRI, Zemer (IL); Nadav MIZRAHI, Tel-Aviv (IL); Viki YELENSKI, Carmiel (IL); Shay YULZARY, Hod-HaSharon (IL)

(73) Assignee: Sensible Medical Innovations Ltd., Netanya (IL)

(21) Appl. No.: 17/612,611

(22) PCT Filed: May 20, 2020
(86) PCT No.: PCT/IL2020/050551

§ 371 Date: Nov. 19, 2021

Related U.S. Application Data
(60) Provisional application No. 62/850,572, filed on May 21, 2019.

(51) Int. Cl. A61B 5/00

(52) U.S. Cl. CPC A61B 5/6831; A61B 5/6822; A61B 2562/164; A61B 5/6842; A61B 2562/04; A61B 5/6823

(57) ABSTRACT

There is provided an apparatus for positioning a front sensor and/or a back sensor across a thorax of a target individual, the apparatus comprising: a back positioning element comprising: a collar sized and shaped for fitting to a shoulder line and base of a back of a neck of the target individual, and an elongated element having a first end region connected to the collar, and a second end region with a location marker set to correspond to a target anatomical feature of the spine of the target individual, wherein when in use, the elongated element is positioned parallel to and over a long axis of a spine on the back of the target individual, and at least one front sensor and at least one back sensor are positioned on the thorax of the patient relative to the back positioning element for transmitting to and/or sensing from the target region.

### RELATED APPLICATION

This application claims the benefit of priority of U.S. Provisional Patent Application No. 62/850,572 filed on May 21, 2019, the contents of which are incorporated herein by reference in their entirety.

### FIELD AND BACKGROUND OF THE PRESENT INVENTION

The present invention, in some embodiments thereof, relates to alignment of sensors and, more particularly, but not exclusively, to systems and methods for alignment of sensors across the chest of a target patient.

Alignment of sensors may affect accurate measurements of target tissues located within the body of a target individual, for example, in measurement systems based on transmission of energy between two sensors located at different parts of the body through the target tissue, or using a single sensor (e.g., receiving signals, transmitting signals, and/or a transceiver that receives reflections of its own signal). Alignment of sensors for accurate measurement may be prone to error in placement of the sensors.

### SUMMARY OF THE PRESENT INVENTION

According to a first aspect, an apparatus for positioning at least one of a front sensor a back sensor at a target region across a thorax of a target individual, comprises: a back positioning element comprising: (i) a collar sized and shaped for fitting to a shoulder line and base of a back of a neck of the target individual, and (ii) an elongated element having a first end region connected to the collar, and a second end region with a location marker set to correspond to a target anatomical feature of the spine of the target individual, wherein when in use, the elongated element is positioned parallel to and over a long axis of a spine on the back of the target individual, and at least one front sensor and at least one back sensor are positioned on the thorax of the patient relative to the back positioning element for transmitting to and/or sensing from the target region.

According to a second aspect, a method for positioning at least one front and at least one back sensor at a target region across a chest of a target individual, comprises: providing the apparatus according to an implementation of the first aspect, positioning the collar of the back positioning element on the shoulder line and/or base of the neck of the target individual, positioning the location marker of the elongated element of the back positioning element at a predefined anatomical landmark s of a spine of the target individual, positioning the arch shaped portion of the holding mechanism over a shoulder of the target individual such that at least one front sensor and at least one back sensor coupled to the holding mechanism are positioned across the thorax, and adjusting a position of a tab element coupled to the elongated front portion of the holding mechanism at a predefined anatomical location on the chest.

In a further implementation of the first and second aspects, further comprising a holding mechanism designed for coupling to the back positioning mechanism, the holding mechanism including: (i) a substantially arc shaped portion for fitting over a shoulder of the target patient, (ii) an elongated front portion for connecting to at least one front sensor for contacting a chest of the target individual, and (iii) an elongated back portion for connecting to at least one back sensor for contacting a back of the target individual, wherein when in use, the location of the holding mechanism is set relative to the positioned back positioning element, and the at least one front sensor and the at least one back sensor are positioned on the thorax of the patient by the holding mechanism relative to the back positioning mechanism for transmitting to and/or sensing from the target region.

In a further implementation of the first and second aspects, further comprising: a non-transitory memory having stored thereon a code that when executed by at least one hardware processor of a computing device causes the at least one hardware processor to: control activation of the at least one back sensor and the at least one front sensor, receive output of the at least one back sensor and the at least one front sensor, and compute an estimate of an amount of fluid within the target tissue of the target individual according to the output of the at least one back sensor and the at least one front sensor.

In a further implementation of the first and second aspects, the back positioning element has an approximately Y shape, the collar having a shape approximately as the short top arms of the Y, and the elongated element of the back positioning element having a shape approximately as the long arm of the Y.

In a further implementation of the first and second aspects, the holding mechanism is shaped as U having a first arm shorter than a second arm, the front portion of the holding mechanism corresponding to the first arm of the U, the back portion of the holding mechanism corresponding to the second arm, and the arc shaped portion of the holding mechanism corresponding to the curve of the U.

In a further implementation of the first and second aspects, the back positioning element further comprise an adjustment mechanism for adjusting a distance between the holding mechanism and the elongated element of the back positioning element.

In a further implementation of the first and second aspects, the adjustment mechanism includes a plurality of stop station selectors arranged in a two dimensional pattern, each stop station selector is set at a predefined location of the holding mechanism for a setting a different distance of the holding mechanism relative to the elongated element of the back positioning element.

In a further implementation of the first and second aspects, each one of the plurality of stop station selectors corresponds to a combination of at least one physical and/or anatomical patient parameters.

In a further implementation of the first and second aspects, the adjustment mechanism adjusts the location of the holding mechanism within a two dimensional plane substantially parallel to the back of the target individual.

In a further implementation of the first and second aspects, the front portion element includes at least one tab element set such that in use when the at least one tab element is positioned at a defined anatomical landmark of the target individual, and the at least one front sensor is positioned relative to the chest at a location corresponding to the target region.

In a further implementation of the first and second aspects, the defined anatomical landmark is selected from the group consisting of: suprasternal notch, clavicle, and sternum.

In a further implementation of the first and second aspects, the holding mechanism, and the back positioning element are sized, shaped, and set for positioning the at least one front sensor and the at least one back sensor across the target region located within at least one member selected from the group consisting of: left lung, right lung, right middle lobe, right upper lobe, right lower lobe, left upper lobe, right lower lobe, heart, trachea, and combinations of the aforementioned.

In a further implementation of the first and second aspects, further comprising at least one front sensor positioning element coupled to the at least one front sensor for adjustment of an orientation of the at least one front sensor for contacting the chest of the target individual corresponding to the target region, the at least one front sensor positioning element coupled to the front portion of the holding mechanism, and/or at least one back sensor positioning element coupled to the at least one back sensor for adjustment of an orientation of the at least one back sensor for contacting a back of the target individual corresponding to the target region, the at least one back sensor positioning element coupled to the back portion of the holding mechanism.

In a further implementation of the first and second aspects, the holding mechanism includes a plurality of rigid sections connected by spring loaded hinges designed for adjustment of a distance between the at least one front sensor and the at least one back sensor and for application of a spring force for urging the at least one back sensor and/or the at least one front sensor to the thorax of the target individual.

In a further implementation of the first and second aspects, the collar is sized and/or shaped to cover an arch of about 40-60 degrees of the back of the neck of the target individual.

In a further implementation of the first and second aspects, the sensors are selected from the group consisting of: electromagnetic (EM) transmitters and/or receivers, ultrasound transmitters and/or receivers, radiofrequency (RF) transmitters and/or receivers, treatment elements, chemical injectors, and imaging elements.

In a further implementation of the first and second aspects, the holding mechanism made up of an elastic material set for increasing an distance between the at least one front sensor and the at least one back sensor for accommodating the thorax and for urging the at least one front sensor and the at least one back sensor towards one another, wherein when in use, the holding mechanism applies a force to the at least one front sensor and the at least one back sensor against the thorax at the position corresponding to the target region.

In a further implementation of the first and second aspects, the apparatus is set for positioning the at least one back sensor between about 2-9 centimeters (cm) medially relative to a long axis of the spine, and between about 8-30 centimeters below an upper end of a vertebra prominence of the target patient.

In a further implementation of the first and second aspects, the sensors are EM transducers and about 30% of the effective EM capture and/or transmission area of the EM transducers is at the set position.

In a further implementation of the first and second aspects, further comprising additional sensors for computing a distance between the front sensor and back sensor when the apparatus is in use.

In a further implementation of the first and second aspects, further comprising transmitting EM energy between the at least one front sensor and the at least one back sensor across the chest and through the target region of the target individual, measuring the EM energy transmitted through the target region, and computing an amount of fluid in the target region.

In a further implementation of the first and second aspects, further comprising selecting one of a plurality of stop stations of the holding mechanism relative to the elongated element of the back positioning element according to one of the plurality of stop stations corresponding to a combination of at least one anatomical and/or physical dimensions of the thorax of the target individual.

In a further implementation of the first and second aspects, the holding mechanism is connected to the back positioning element by a connector that is inserted into the one of the plurality of stop stations.

Unless otherwise defined, all technical and/or scientific terms used herein have the same meaning as commonly understood by one of ordinary skill in the art to which the present invention pertains. Although methods and materials similar or equivalent to those described herein can be used in the practice or testing of embodiments of the present invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the present invention can involve performing or completing selected tasks manually, automatically, or a combination thereof. Moreover, according to actual instrumentation and equipment of embodiments of the method and/or system of the present invention, several selected tasks could be implemented by hardware, by software or by firmware or by a combination thereof using an operating system.

For example, hardware for performing selected tasks according to embodiments of the present invention could be implemented as a chip or a circuit. As software, selected tasks according to embodiments of the present invention could be implemented as a plurality of software instructions being executed by a computer using any suitable operating system. In an exemplary embodiment of the present invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the present invention are herein described, by way of example only, with reference to the accompanying drawings. With specific reference now to the drawings in detail, it is stressed that the particulars shown are by way of example and for purposes of illustrative discussion of embodiments of the present invention. In this regard, the description taken with the drawings makes apparent to those skilled in the art how embodiments of the present invention may be practiced.

In the drawings:

FIG. 1 is a block diagram of an apparatus for positioning front and/or back sensor(s) across a thorax of a target individual for transmitting energy to and/or receiving energy from a target region of the target individual, in accordance with some embodiments of the present invention;

FIG. 2 is an exemplary implementation of an apparatus for positioning front sensor(s) and/or back sensor(s) across a thorax of a target patient, in accordance with some embodiments of the present invention;

FIG. 3 is a schematic of holding mechanism alone, without being connected to the back positioning element, in accordance with some embodiments of the present invention;

FIG. 4 is a schematic presenting a lateral view of the apparatus when not in use, in accordance with some embodiments of the present invention;

FIG. 5 is a schematic of the apparatus, where holding mechanism is depicted at a maximum distance and/or when in use, between front sensor and back sensor, in accordance with some embodiments of the present invention;

FIG. 6 is a schematic of back positioning element, in accordance with some embodiments of the present invention;

FIG. 7 is a schematic of the apparatus, where the holding mechanism is attached to the back positioning element via connectors, in accordance with some embodiments of the present invention;

FIG. 8 is a schematic of the holding mechanism of the apparatus, shown independently of the back positioning element, in accordance with some embodiments of the present invention;

FIGS. 9A-9C are schematics and/or images depicting an example of the positioning apparatus on a target individual, in accordance with some embodiments of the present invention;

FIG. 10 is a flowchart of a method for positioning the front and/or back sensor(s) across a thorax and at a target region of a target individual, in accordance with some embodiments of the present invention;

FIG. 11 is a schematic of an exemplary implementation of a collar used for height positioning in a back positioning mechanism, in accordance with some embodiments of the present invention; and

FIG. 12 is a schematic of a close up view of a collar used for height positioning in a back positioning mechanism, in accordance with some embodiments of the present invention.

### DESCRIPTION OF SPECIFIC EMBODIMENTS OF THE PRESENT INVENTION

The present invention, in some embodiments thereof, relates to alignment of sensors and, more particularly, but not exclusively, to systems and methods for alignment of sensors across the chest of a target patient.

An aspect of some embodiments of the present invention relates to an apparatus for positioning a front and/or back sensor across a target region of a thorax of a target individual, for example, electromagnetic (EM) transceivers for sensing fluid within a lung of a patient (or other examples as described herein). The apparatus includes elements that are designed to be positioned at corresponding anatomical landmark(s). The apparatus is set so that when the elements are at the anatomical landmark(s), the front and/or back sensors are correctly positioned for sensing the target region of the thorax. The apparatus includes a back positioning element that includes a collar designed to be applied in the anatomical location of the shoulder line/base of the back of the neck, and an elongated element with a location marker at its bottom region (e.g., a notch), designed for positioning on the spine (at the back, for example, at a certain vertebra) of the target individual, for example, in parallel and/or over a long axis of the spine, and/or over a selected anatomical feature of the spine such as a certain vertebra. Optionally, the distance of the back sensor from the anatomical feature of the spine closest to the back sensor is set. When the back positioning element is set in place, an optional holding mechanism coupled to the back positioning element is at a predefined location where a back and/or front sensor attached to the holding mechanism are positioned across the thorax of the patient corresponding to the target region. The holding mechanism may be approximately U shaped, optionally including a substantially arc shaped element designed to fit over the shoulder of the patient, an elongated front portion for connecting to the front sensor(s), and an elongated back portion for connected to the back sensor(s). The sensors may be activated for transmitting energy to and/or sensing energy from the target region, for example, EM energy to the lung for computing an amount of fluid in the lung. Alternatively, no holding mechanism is used, and an operator may manually hold the sensor(s) in place according to relative locations between the sensor(s) and the back positioning element. In another implementation, the operator place a marking (e.g., sticker, made using a marker) on the thorax of the patient according to relative locations between the sensor(s) and the back positioning element, and then places the sensor(s) on the marking with a different mechanism holding mechanism and/or manually.

Optionally, when the tab element(s) and/or collar and/or location marker on the elongated element of the back positioning element are placed at their respective target anatomical locations (as described herein), the front sensor and/or the back sensors are within about 2, 3, 4, 6, 8 or other value or ranges using the cited values or other values, of their target locations corresponding to the target region for sensing and/or treating.

Optionally, the apparatus includes a tab element coupled to the front portion of the holding mechanism. The tab element is set so that when in use and the tab element is positioned at the suprasternal notch of the patient, the front sensor is located at the correct anatomical location of the patient for transmitting to and/or receiving signals from the target region.

Alternatively or additionally, the apparatus includes two tab elements (or other measured distance indicator or pointer) elements coupled to the front portion of the holding mechanism and/or front sensor. The tab elements are set so that when positioned simultaneously at the clavicle and the sternum, the front sensor is located at the correct anatomical location of the patient for transmitting to and/or receiving signals from the target region. Optionally, the apparatus is set to apply a force that urges the sensors towards the thorax of the patient, for example, a system of rigid elements connected by springs.

Optionally, the position of the holding mechanism relative to the back positioning element is adjustable, optionally within a 2D plane substantially parallel to the back of the target individual, to accommodate different sizes of thoraxes.

Optionally, the back positioning element includes a collar designed to be positioned along the neck and/or one or both sides of the neck and/or shoulder(s). The collar is shaped to fit the target anatomical location. Optionally, the back positioning element includes a collar shaped to fit the target anatomical landmark of the shoulder line and/or base of the back of the neck. A component of the collar may be sized and/or shaped to fit the sides of the neck to help make sure the collar fits symmetrically around the neck. The collar is optionally a part of the back positioning element (which sets the vertical alignment of the sensors via the holding element), and sets the height of the back positioning element in relation to the shoulder line/base of the back of the neck.

The tab element and/or the location marker at the bottom of the elongated element, and/or collar (sometimes collectively referred to herein as positioning elements), are designed to be applied in predefined anatomical locations on the thorax of the target individual (optionally along the suprasternal notch, the spine, and the base of the back of the neck, respectively, as described herein, for example, according to visual and/or mechanical matching of the respective anatomical positioning element to the anatomical landmark. The positioning elements and the mechanical elements (i.e., holding mechanism and back positioning element attached sensors) are mechanically coupled, such that when the positioning elements are in a proper anatomical position the sensors attached to the holding mechanism, are in proper desired positions on the thorax with respect to target regions. When the positioning elements (i.e., one or more of: tab element and/or the location marker at the bottom of the elongated element, and/or collar) are in place, the front and/or back sensors are at the correct locations, with the holding mechanism being in one or more slightly different orientations. The mechanical coupling of the holding mechanism and back positioning element may be adjusted (e.g., distances and/or orientation) such that the sensors positions are adjusted relative to the positioning elements.

The holding element may be adapted such that the distance and/or relative position of the sensors may be changed, as described herein. The holding element may exert a force to better attach the sensors to the thorax, as described herein.

An aspect of some embodiments of the present invention relates to a method for positioning front and/or back sensors at a target region across a chest of a target individual. The apparatus as described herein may be provided. The back positioning element is positioned on the back of the patient according to the collar positioned on the shoulder line and/or base of the neck and the location marker at the bottom of the elongated element positioned at a target anatomical location of the spine. At the same time, since the component of the apparatus are connected, the elongated element of the back position mechanism of the apparatus is positioned in parallel to and over a long axis of a spine of the target individual. The arc shaped portion of the holding mechanism is positioned over a shoulder of the target individual. The front and/or back sensors are positioned across the thorax. A tab element coupled to the elongated front portion of the holding mechanism is positioned at the suprasternal notch, such that the front sensor(s) coupled to the elongated front portion is further adjusted across the chest of the target individual to reach a target location on the chest of the individual. The sensors and/or treatment elements may be activated for transmitting energy to and/or receiving energy from the target region, for example, EM energy to the lung and/or focused ultrasound for ablation, and/or other options as described herein. Received signals may be processed, for example, computing an amount of fluid in the lung. In another example, signals may be computed for adjusting the treatment elements, such as adjustment of the focused ultrasound. The patient may be diagnosed and/or treated (e.g., based on the diagnosis and/or treated by energy emitted by the sensors) according to the processed signals, for example, treated for pulmonary edema, and/or application of energy by the sensors.

As used herein the term sensor may sometimes be interchanged with, and/or may sometime include the term treatment application element.

At least some implementations of the apparatus and/or method described herein address the technical problem of positioning sensors across the thorax of a patient, optionally improving accuracy of correctly positioning the sensors and/or treatment application elements (e.g., applicators of energy for direct treatment, focused ultrasound, RF energy, radiation therapy applicator, and/or for applying or administering or extracting substances for example via a syringe or transdermal application, and/or for imaging probes). Optionally while applying pressure to urge the sensors when contacting the thorax. The apparatus may be repeatedly applied to different patients (optionally of different sizes) for accurate positioning of the sensors. The apparatus may be repeatedly applied to the same patient at different sessions, for accurate repeated positioning of the sensors at the same location (within a tolerance range) for each session. The technical problem may relate to rapidly and/or easily and/or accurately positioning of sensors across the thorax for obtaining measurements for treatment (and/or for diagnosis and planning of treatment based on the diagnosis and/or for monitoring treatment) of the patient, for example, estimating an amount of fluid in one or both lungs of the patient.

At least some implementations of the apparatus and/or method described herein addresses the medical problem of treatment of a patient, for example, by application of energy for direct treatment of a target region (e.g., focused ultrasound, RF energy, radiation applicator) and/or treatment selected and/or guided based on sensor data obtained from a thorax of a patient, optionally, based on EM radiation (or other energy such as ultrasound or electrical currents) transmitted into tissues within the thorax, for example, fluid in lung(s) or other tissues. The medical problem may relate to increasing the accuracy of the sensed data used for treatment of the patient. Another medical problem may relate to improving the process of obtaining the sensed data. The medical problem is addressed by the apparatus, which is easy to accurately position on the thorax to collect the medical data used for treatment of the patient.

At least some implementations of the apparatus and/or method described herein address the technical problem by the holding mechanism and back positioning element that are designed for being set at one or more anatomical landmarks of the patient, in an accurate and/or repeatable manner. In at least some implementations, the height of the apparatus in the back is set relative to the base of the back of the neck and laterally on the back relative to the spine of the patient and relative to the suprasternal notch of the patient on the front. Once predefined elements of the apparatus are positioned as described herein, the front and/or back sensors (e.g., EM transceivers) may be set at their correct locations relative to the anatomical landmarks for sensing a target region within the body of the patient, for example, the lungs.

Before explaining at least one embodiment of the present invention in detail, it is to be understood that the present invention is not necessarily limited in its application to the details of construction and the arrangement of the components and/or methods set forth in the following description and/or illustrated in the drawings and/or the Examples. The present invention is capable of other embodiments or of being practiced or carried out in various ways.

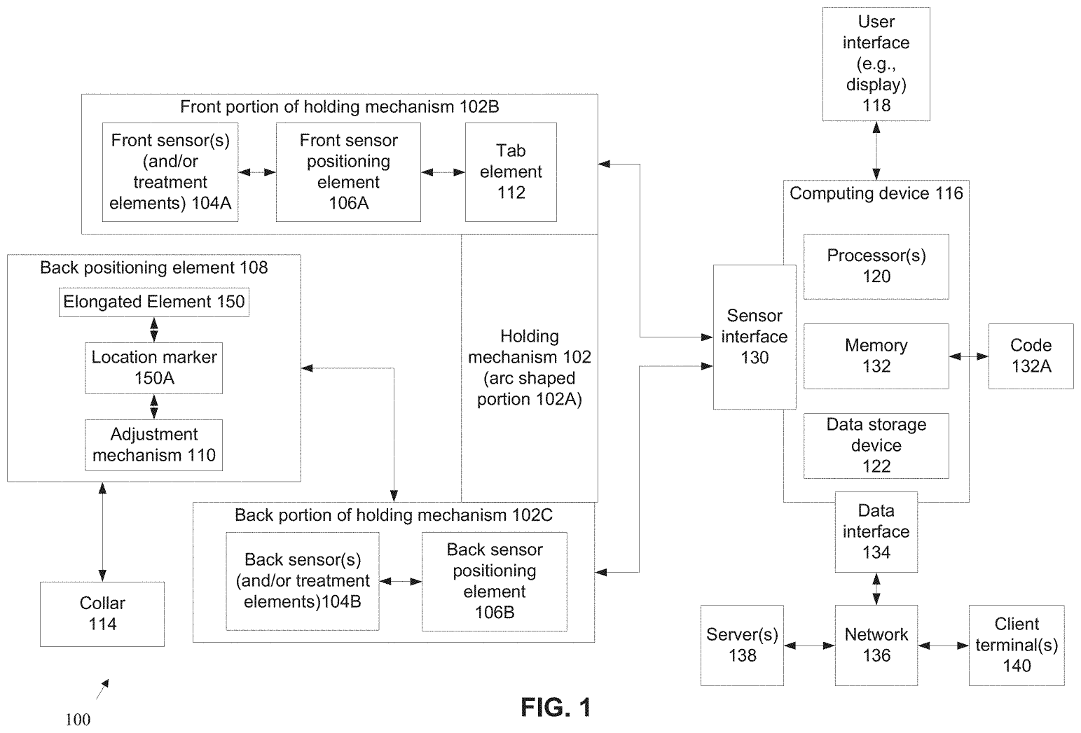

Reference is now made to FIG. 1, which is a block diagram of an apparatus 100 (and/or system 100) for positioning front and/or back sensor(s) across a thorax of a target individual for transmitting energy to and/or receiving energy from a target region of the target individual, in accordance with some embodiments of the present invention.

Apparatus 100 includes a back positioning element 108, a collar 114, and a location marker located on a bottom region of an elongated element 150 of back positioning element 108, as described herein in additional detail.

Optionally, the back positioning element 108 has an approximately Y shape, with the collar having a shape approximately as the short top arms of the Y, and the elongated element 150 of the back positioning element 108 having a shape approximately as the long arm of the Y.

Optionally, apparatus 100 includes a holding mechanism 102 that connects (e.g., mechanically clipped to, using magnets, an integrated component) to back positioning mechanism 108, as described herein.

Holding mechanism 102, shaped approximately U shaped, includes a substantially arc shaped portion 102A (for fitting over a shoulder of the target patient) an elongated front portion 102B for connecting to at least one front sensor(s) 104A for contacting a chest of the target individual, and an elongated back portion 102C for connecting to at least one back sensor 104B for contacting a back of the target individual. Optionally, both the position and/or orientation of both front sensor(s) 104A and back sensor(s) 104B are adjustable. Alternatively, the position and/or orientation of one sensor is fixed, while the position and/or orientation of the other sensor is adjustable, for example front sensor is fixed and back sensor is adjustable, or front sensor is adjustable and back sensor is fixed.

A front sensor positioning element 106A, may couple front sensor(s) 104A to front portion 102B. A front sensor positioning element 106A may be designed to adjust the distance and/or orientation of front sensor(s) 104A, in one or more degrees of freedom (e.g., six), for example implemented as an extendible part and/or a gimbal. A back sensor positioning element 106B, may couple back sensor(s) 104B to back portion 102C. A back sensor positioning element 106B may be designed to adjust the distance and/or orientation of back sensor(s) 104B, in one or more degrees of freedom (e.g., six), for example implemented as a gimbal. Front and/or back sensor positioning elements 106A-b may adjust the distance and/or orientation of respective front and/or back sensors 104-B to conform to the shape of the surface of the thorax they come in contact with.

Exemplary sensor 104A and/or 104B include EM transceivers, and ultrasound transmitters. Alternatively or additionally, sensor 104A and/or 104B are interchanged and/or combined with treatment elements. Exemplary sensors and/or treatment elements 104A-B include: treatment application elements, applicators of energy for direct treatment, focused ultrasound by ultrasound transmitters, RF energy by RF transmitters, radiation therapy applicator by a radiation element, and/or for applying or administering or extracting substances for example via a syringe or transdermal application, and/or for imaging probes.

Sensors 104A and/or 104B may be connected to holding mechanism 102 using manually and/or automatically extending arms (e.g., front and back sensor positioning elements 106A-B) for better conforming and/or fitting to a patient thorax. The extending arms may optionally include a measurement element (e.g., a ruler, sensor) to allow for the quantification of the extension to assess its effect on the positioning of the sensor.

Sensors 104A and/or 104B may be extended out from their respective housing towards the body of the subject to create an effect of increasing the attachment force, for example, urged by a spring.

Front portion 102B (or front sensor positioning element 106A) may include a tab element 112. Tab element 112 is set such that when in use, when the tab element contacts a suprasternal notch of the target individual, the front sensor 104A (or front sensor positioning element 106A) is further adjusted relative to the chest to at a location corresponding to the target region. Tab element 112 helps correctly further adjust the position of front sensor 104A.

Back positioning element 108 includes collar 114 at a caudal region thereof designed to be applied to an anatomical location of the shoulder line and/or base of the back of the neck, and/or an elongated element 150 with a location marker 150A for positioning at a defined anatomical landmark, location parallel to and over a long axis of a spine on the back of the target individual. When in use back positioning element 108 is set to position the back portion of holding mechanism 102C at a defined location relative to the target individual, with the front sensor 104A and/or back sensor 104B correctly located for transmitting energy to and/or sensing energy from the target region. When in use, collar 114 set the height of the back positioning element 108 and by proxy the height of the back of the holding mechanism 102 and by proxy the back sensor 104B.

Location marker 150A may be implemented as, for example a notch, a hole, a marking, and/or other visual visible and/or physically unique feature of elongated element 150 of back positioning element 108.

Back positioning element 108 may include an adjustment mechanism 110 for adjusting the position of holding mechanism 102 relative to back positioning element 108, optionally within a two dimensional plane (e.g., continuously and/or step wise) substantially parallel to the back of the target individual. The adjustment is performed to accommodate different sized of thoraxes such that the front and/or back sensors are located at the correct target region for the different sized thoraxes. Adjustment mechanism 110 may be integrated within elongated element 150, as described herein.

Holding mechanism 102 and back positioning element 108 are sized, shaped, and set for positioning front sensor 104A and/or back sensor 104 across the target region located within, for example: left lung, right lung, right middle lobe, right upper lobe, right lower lobe, left upper lobe, right lower lobe, heart, trachea, and combinations of the aforementioned.

Apparatus 100 may be used for achieving correct positioning of applicators, probes, sensors and the like in any position on the thorax of the subject, including for example on the left side of the subject thorax.

The positioning of apparatus 100 on the thorax allows for the positioning of external elements in relation to internal organs in the body of the subject. For example, in relation to the right lung, left lung, heart, trachea, etc. The positioning may be designed such that specific organs are avoided, for example the diaphragm, the spine, the clavicle bone, etc.

Variations of apparatus 100 include, for example, substitution and/or inclusion of applicators and/or treatment elements and/or probes in place or and/or in addition to front and/or back sensors, a single sensor at the front and/or back, multiple sensors at the front and/or back.

Additional optional components that may be connected to apparatus 100 are now described.

Optionally, apparatus 100 includes and/or is in communication with a computing device 116, for example, one or more of: a server, a computing cloud, a mobile device, a desktop computer, a virtual machine, a virtual server, a computing cloud, a thin client, a Smartphone, a Tablet computer, a laptop computer, a wearable computer, glasses computer, and a watch computer. Computing device 116 may be integrated (entirely or some components and/or features) into apparatus 100. Computing device 116 may be implemented as an add-on to an existing workstation and/or other devices, for example, for presenting a GUI created from processing of the sensor data.

Computing device 116 may receive the output of sensors 104A and/or B (e.g., raw signals and/or processed signals such as digital signal converted from analogue measurements made by the sensors), optionally via one or more sensor interfaces 130, for example, a wire connection (e.g., physical port, for example, a cable connecting the sensors to the port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)).

Apparatus 100 may be made up of materials including, for example: plastic, metal, rubber parts, Nylon, silicone, PU polyurethane, Polycarbonate, ABS, ABS (separate from PC), PC-ABS, and combinations of the aforementioned, depending for considerations of mechanical properties like rigidity, weight, durability, and the like.

Computing device 116 may control energy transmission by sensor 104A and/or 104B, for example, by sending signals via sensor interface 130. Sensor interface may optionally include an electronic module for generating and/or receiving and/or processing analogue or digital sensor signals.

Computing device 116 may include locally stored software (e.g., code 132A stored in a memory 132 that when executed by hardware processor(s) 120) performs one or more of the computational features described herein and/or may act as one or more servers (e.g., network server, web server, a computing cloud, virtual server) that provides services (e.g., one or more of the acts described with reference to FIG. 1) to one or more client terminals 140 and/or one or more server 138, for example, central processing of remotely collected sensor data. Such services may be provided over a network 136, for example, providing software as a service (SaaS) to the client terminal(s) 140, providing an application for local download to the client terminal(s) 140, as an add-on to a web browser and/or other sensor processing application, and/or providing functions using a remote access session to the client terminals 208, such as through a web browser, application programming interface (API), and/or software development kit (SDK).

Optionally, computing device 116 controls activation of front sensor(s) 104A and/or back sensor(s) 104B and/or receives output of front sensor(s) 104A and/or back sensor(s) 104B, and computes an estimate of an amount of fluid within the target tissue (e.g., lung) of the target individual according to the output.

Hardware processor(s) 120 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 120 may include one or more processors (homogenous or heterogeneous), which may be arranged for parallel processing, as clusters and/or as one or more multi core processing units.

Memory 132 (also referred to herein as a program store, and/or data storage device) stores code instruction for execution by hardware processor(s) 120, for example, a random access memory (RAM), read-only memory (ROM), and/or a storage device, for example, non-volatile memory, magnetic media, semiconductor memory devices, hard drive, removable storage, and optical media (e.g., DVD, CD-ROM). For example, memory 132 may store code 132A that implement one or more computational features described herein.

Computing device 116 may include a data storage device 122 for storing data, for example, the received signals from the sensors. Data storage device 122 may be implemented as, for example, a memory, a local hard-drive, a removable storage device, an optical disk, a storage device, and/or as a remote server and/or computing cloud (e.g., accessed over network 136).

Computing device 116 may include data interface 134, optionally a network interface, for connecting to network 136, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 116 may access one or more remote servers 138 using network 136, for example, to obtain an update of code 132A, and/or to obtain other data (e.g., patient medical data from the EMR of the patient).

It is noted that sensor interface 130 and data interface 134 may be implemented as a single interface (e.g., network interface, single software interface), and/or as two independent interfaces such as software interfaces (e.g., as APIs, network ports) and/or hardware interfaces (e.g., two network interfaces), and/or combination (e.g., single network interface, and two software interfaces, two virtual interfaces on a common physical interface, virtual networks on a common network port). The term/component sensor interface 130 may sometimes be interchanged with the term data interface 134.

Computing device 116 may communicate using network 134 (or another communication channel, such as through a direct link (e.g., cable, wireless) and/or indirect link (e.g., via an intermediary computing device such as a server, and/or via a storage device) with one or more of: server(s) 138, client terminal(s) 140, and/or the sensors, and/or other devices, for example, according to different architectural implementations described herein.

Computing device 116 includes and/or is in communication with a user interface 118 that includes a mechanism designed for a user to enter data (e.g., enter patient identity), and/or for a user to view data (e.g., analysis of signals collected by the sensor(s)). Exemplary user interfaces 116 include, for example, one or more of, a touchscreen, a display, a keyboard, a mouse, augmented reality glasses, virtual reality glasses and voice activated software using speakers and microphone. User interface 116 may include a graphical user interface (GUI) presented on a display designed for the user to enter the data and/or view the data.

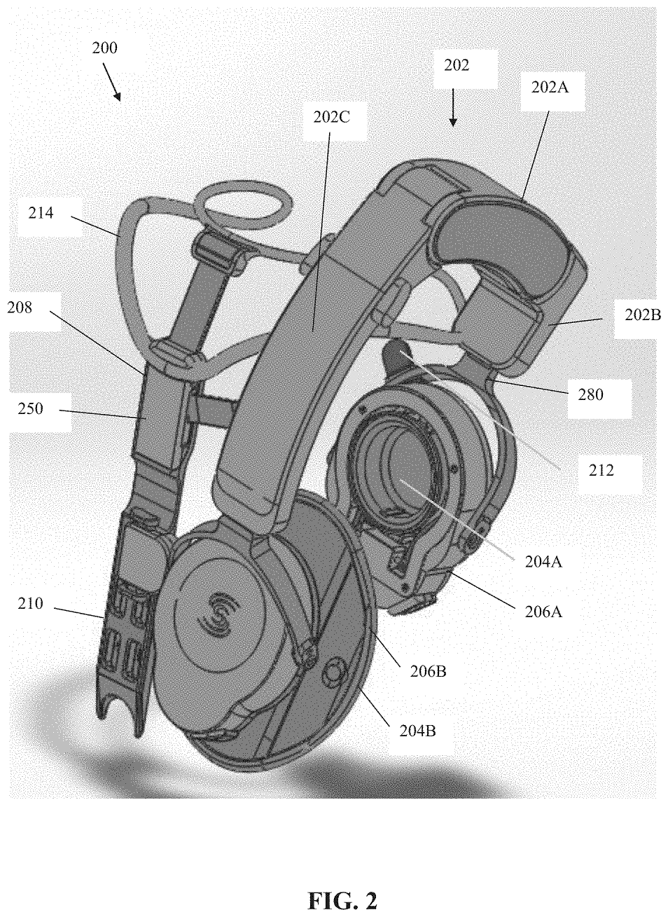

Reference is now made to FIG. 2, which is an exemplary implementation of an apparatus 200 for positioning front sensor(s) 204A and/or back sensor(s) 204B across a thorax of a target patient, in accordance with some embodiments of the present invention. Components of apparatus 200 may be based on, combined with, and/or replaced with components of apparatus 100 described with reference to FIG. 1.

Apparatus 200 includes a holding mechanism 202 that includes an arc shaped portion 202A, an elongated front portion 202B, and an elongated back portion 202C, as described herein. Holding mechanism 202 is shaped as U, having a first arm shorter than a second arm. Front portion 202B of holding mechanism 202 corresponds to the first arm of the U. Back portion 202C corresponds to the second arm. Arc shaped portion 202A corresponds to the curve portion of the U.

An optional front sensor positioning element 206A couples front sensor 204A to front portion 202B. Front sensor positioning element 206A is set for adjustment of an orientation of front sensor 204A for contacting the chest of the target individual corresponding to the target region, for example, including a gimbal. An optional back sensor positioning element 206B couples back sensor 204B to back portion 202C. Back sensor positioning element 206B is set for adjustment of an orientation of back sensor 204B for contacting the back of the target individual corresponding to the target region, for example, including a gimbal.

Optionally, front sensor positioning element 206A includes a tab element 212, as described herein.

Apparatus 200 includes a back positioning element 208, which includes an elongated element 250, an optional adjustment mechanism 210, and an optional collar 214, as described herein.

Apparatus 200 may include an optional extendible arm(s) 280 for better conforming and/or fitting to a patient thorax, as described herein.

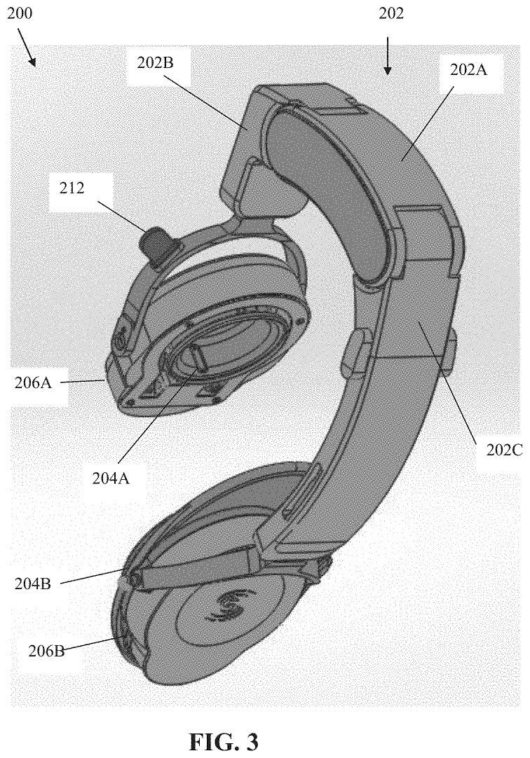

Reference is now made to FIG. 3, which is a schematic of holding mechanism 202 alone, without being connected to the back positioning element, in accordance with some embodiments of the present invention.

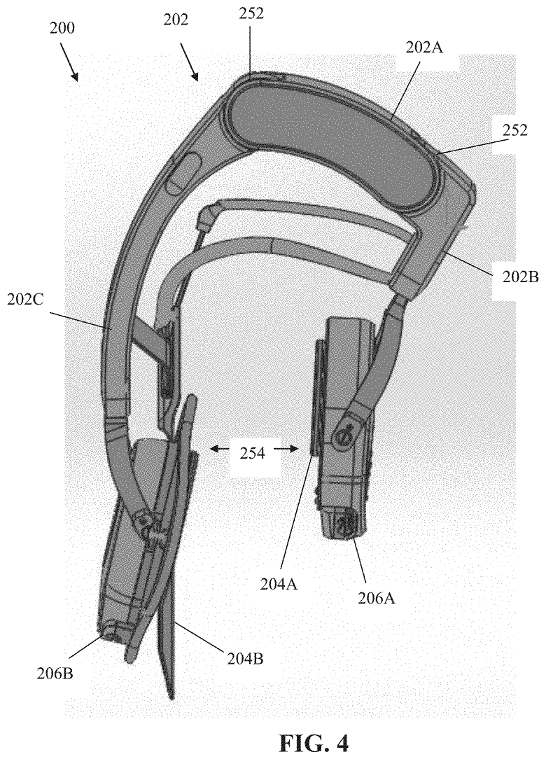

Reference is now made to FIG. 4, which is a schematic presenting a lateral view of apparatus 200 when not in use, in accordance with some embodiments of the present invention. Holding mechanism 202 includes a plurality of rigid sections (e.g., arc shaped portion 202A and/or elongated front portion 202B and/or elongated back portion 202C) connected by spring loaded hinges 252 designed for adjustment of a distance 254 between the front sensor(s) 204A and the back sensor(s) 204B, and optionally for application of a spring force for urging the back sensor(s) 204B and/or the front sensor(s) 204A towards the thorax of the target individual. Alternatively or additionally, holding mechanism 202 is set to be biased for urging front sensor(s) 204A and/or front sensor positioning element 206A, and back sensor(s) 204B and/or back sensor positioning element(s) 206B towards one another, for example, holding mechanism 202 is made from a material having an elastic property where holding mechanism 202 may be spaced apart to increase distance 254 to accommodate the thorax of the individual, for example, metal with elastic properties, plastic combined with springs, and/or rubber. When in use, when distance 254 is increased to accommodate the thorax, holding mechanism 202 applies a force for urging front sensor(s) 204A and/or front sensor positioning element 206A, and back sensor(s) 204B and/or back sensor positioning element(s) 206B towards one another, which urges front sensor(s) 204A against the chest and back sensor(s) 204B against the back.

Alternatively or additionally, front sensor positioning element 206A and/or back sensor positioning element 206B may include a mechanism (e.g., spring, inflatable member) for extending respective sensors 204A-B out from a housing toward the thorax to create an effect of increasing the contact force.

Optionally, elongated front portion 202B and/or elongated back portion 202C include multiple rigid sections connected by hinges, optionally spring loaded. Such design may better adapt to accommodate the shape and/or size of the thorax.

Other variations in architectural design may include, for example,: less or more number of rigid sections, other mechanical constructions allowing variation of the distances 254 and/or forces, for example, use of tightening straps exerting force between the sensors either directly and/or via the holding mechanism, and/or use of lead screw in a clamp like design.

The force applied via elements 202B-C of holding mechanism 202 may be applied to achieve proper contact with the thorax (for example for effective coupling of energy into and/or out of the thorax, e.g. electromagnetic, electrical currents, ultrasound etc.), and/or for achieving a stable application, where apparatus 200 is applied and stays in place on the thorax.

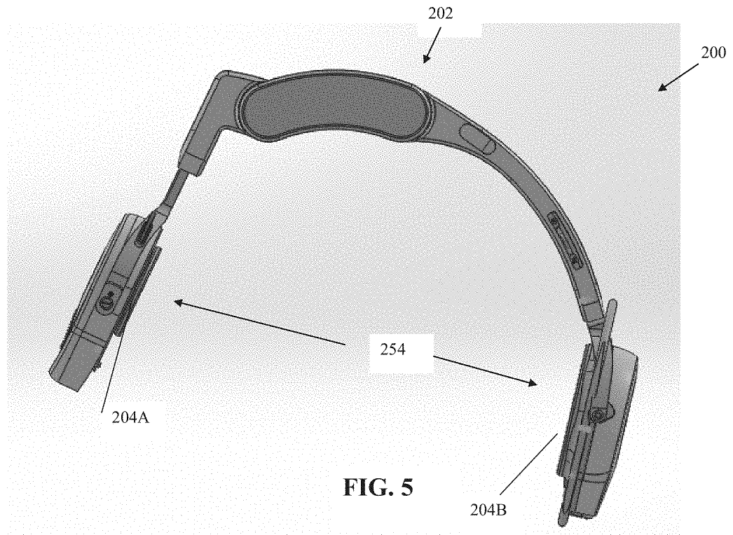

Reference is now made to FIG. 5, which is a schematic of apparatus 200, where holding mechanism 202 is depicted at a maximum distance 254 and/or distance 254 when in use, between front sensor 204A and back sensor 204B, in accordance with some embodiments of the present invention. It is noted that the maximal distance is not necessarily reached when in use in most cases.

Optionally, apparatus 202 includes one or more elements for computing distance 254 between front sensor 204A and back sensor 204B when the apparatus is in use. Exemplary elements for computing distance 254 include: automatically based on sensors located in the holding mechanism, direct measurement of the circumferential distance, based on information gained from angle or linear measurement sensors (e.g., using potentiometer based or optical encoders located in the hinges of the multi-section hinged design or extendible arms described herein), the holding mechanism may include adjustable parts (e.g., including extending elements) allowing for the adjustment of the position and/or orientation of the sensor relative to the holding mechanism in a measurable way.

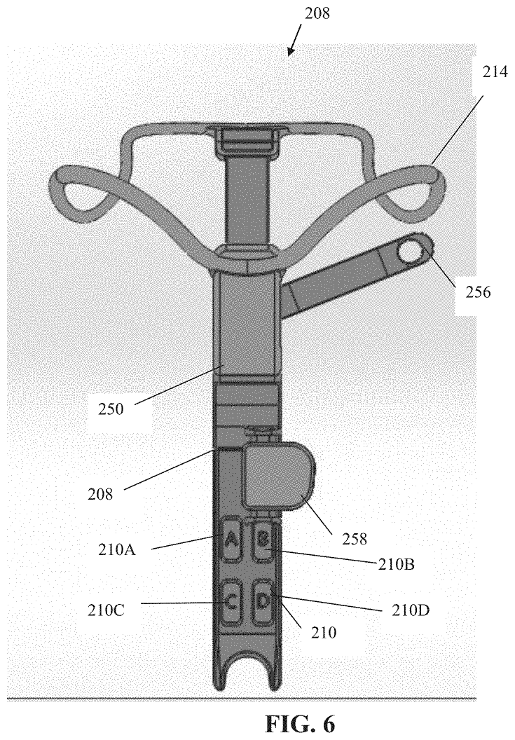

Reference is now made to FIG. 6, which is a schematic of back positioning element 208, in accordance with some embodiments of the present invention.

Back positioning element 208 includes adjustment mechanism 210 for adjusting a relative position between back portion of holding mechanism not shown in FIG. 6) and back positioning element 208. Holding mechanism 202 may connect to adjustment mechanism 210 via a connector 258.

Adjustment mechanism 210 may be designed for adjusting the location of back portion of holding mechanism 202C within a dimension substantially parallel to the back of the target individual and/or within a two dimensional plane substantially parallel to the back of the target individual. Optionally, adjustment mechanism 210 is implemented as multiple stop station selectors 210A-D arranged in a two dimensional pattern parallel to the back of the target individual, for example, shown as “A” 210A, “B” 210B, “C” 210C, and “D” 210D. It is noted that connector 258 is depicted as being connected into a fifth stop station selector. Each stop station selector 210A-D (and the one not shown) is set at a predefined location on the elongated portion 250 of back positioning element 208 for a setting a different relative position of back portion of holding mechanism 202C relative to back positioning element 208. The locations of the stop selectors may be selected, for example, to accommodate different populations of patients having thicker (measured anterior-posterior) and/or longer (measured along the head-feet direction) thoraxes.

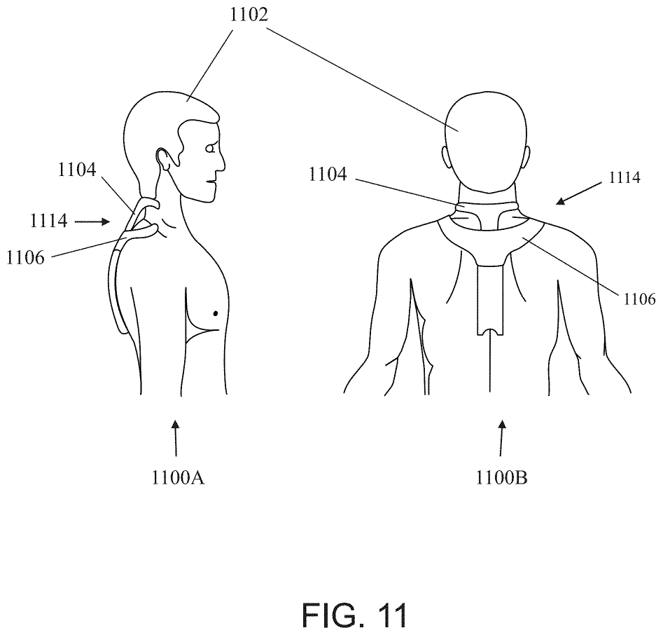

A caudal region of back positioning 208 (i.e., towards the head) is connected to collar 214 set to fit on the back of the base of the neck and/or shoulder line of the target individual, as described herein. Collar 214 is sized and/or shaped to fit the target anatomical location of the back of the base of the neck and/or shoulder line. Optionally, collar 214 is made from a substantially rigid material, for example, plastic and/or metal, for example, as described in additional detail with reference to FIG. 11.

In use, back positioning element 208 is positioned on the back of the target individual by matching a location marker of elongated element 250 to the target anatomical on the spine, and/or collar 214 to the target anatomical location on the back of the neck. To position vertically, back positioning element 208 is applied on the back of the patient where the collar shaped rigid and/or semi-rigid element 214 is positioned on one or two sides of the neck and/or over the shoulder(s) of the subject and resting on them. Collar 214 is set for positioning back positioning element 208 in relation to the base of the neck/shoulder line. For correct lateral positioning, elongated element 250 is aligned with the spine, optionally using a notch line spine alignment element, allowing for concurrent location of the spine by touch and alignment of back positioning element 208. Alternatively to a notch, aligning with the spine can done with a hole in the back positioning element 208 where a finger may fit to feel for the spine vertebrae, and/or a pointing marker.

In another embodiment, the lateral positioning of the back positioning element is achieved solely using a symmetric collar element (e.g., using an upper portion element such as 1104), that when positioned at the anatomical landmark centers the back positioning element.

Another adjustable connector 256 is designed to connect to holding mechanism 202 to further stabilize holding mechanism 202 relative to back positioning element 208.

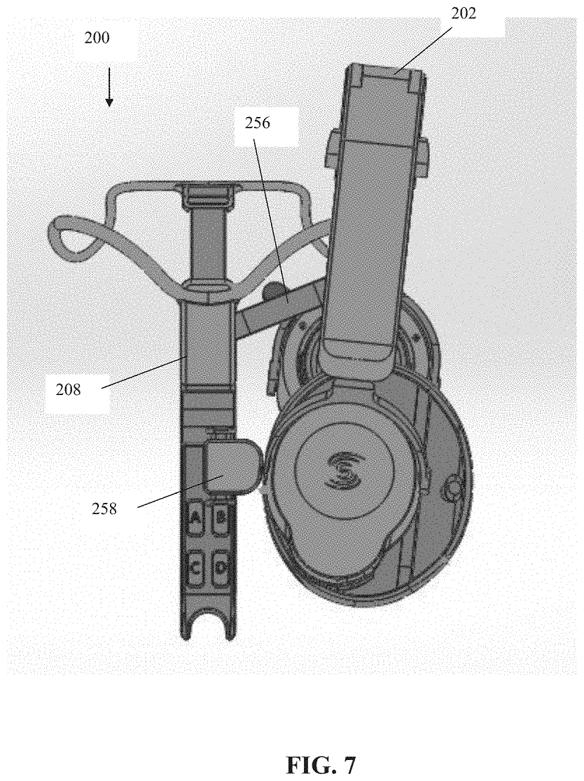

Reference is now made to FIG. 7, which is a schematic of apparatus 200, where holding mechanism 202 is attached to back positioning element 208 via connectors 256 and 258, in accordance with some embodiments of the present invention. Connectors 256 and 258 may be adjustable for adjusting location and/or orientation of holding mechanism 202 relative to back positioning element 208, as described herein. Connectors 256 and 258 may form a permanent connection, or may be detachable, for example, using a click-in mechanism.

Optionally, the back positioning element 208 may be detached and/or detachable from holding mechanism 202 and the correct positioning of sensors 204A-B may be achieved by separately applying positioning mechanism 208 on the thorax of the target individual according to the anatomical markers described herein (e.g., collar to the base of the back of the neck), and positioning holding mechanism 202 back positioning element in relation to back positioning element 208 by mechanically coupling holding mechanism 202 to back positioning element 208 as described herein, and/or visually aligning markers on holding mechanism 202 with markers on back positioning element 208.

The phrase mechanical coupling as used herein may sometimes refer to using the elements (e.g., connectors) described herein by which the back positioning element is used to set a position on the thorax and the holding mechanism is positioned in a location relative to the back positioning element, implemented for example, by a mechanical connection, and/or by alignment of elements, where the holding mechanism and the back positioning element do not necessarily come in physical contact.

The coupling of the back positioning element to the holding mechanism that includes the sensors, optionally positions the back sensor in relation to the back positioning element so as to achieve a specific predefined position of the back sensor in relation to the anatomical landmarks.

The mechanical coupling may be adjusted to achieve different, defined and/or quantifiable, positions of the sensor in relation to the anatomical landmarks using adjustment mechanism 210.

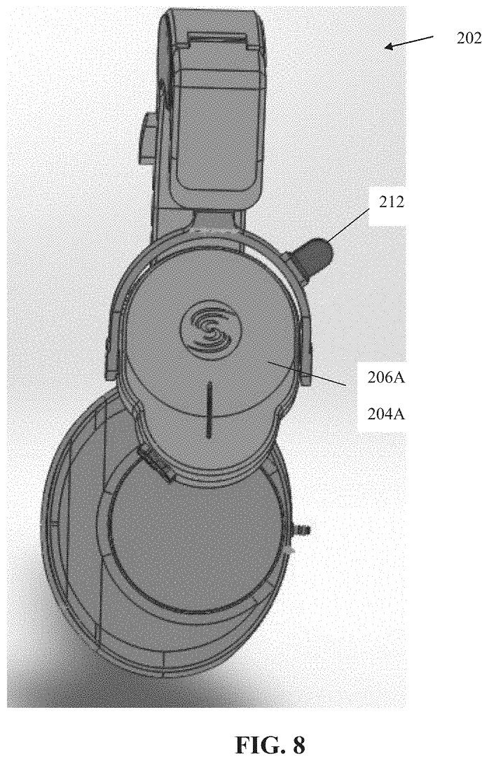

Reference is now made to FIG. 8, which is a schematic of holding mechanism 202 of apparatus 200, shown independently of the back positioning element, in accordance with some embodiments of the present invention. As depicted, tab element 212 is mechanically coupled to (e.g., connected to a front side of) front sensor positioning element 206A connected to front sensor 204A. As described herein, the location of tab element 212 is set so that when in use, when tab element 212 is positioned at the suprasternal notch of the target individual, front sensor 204A is positioned at the correct location for transmitting energy to and/or receiving energy from the target region of the patient.

Reference is now made to FIG. 11, which is a schematic of an exemplary implementation of a collar 1114 used for height positioning in a back positioning element, in accordance with some embodiments of the present invention. Collar 1114 is connected to a part of the back positioning element, optionally to the upper (i.e., caudal direction) region thereof, as described herein. Schematic 1100A is a side view of a subject 1102 wearing collar 1114. Schematic 1100B is a back view of subject 1102 wearing collar 1114. Collar 1114 includes an upper portion 1104 made of a relatively more flexible material than a lower portion 1106, which is made of a relatively more rigid material. Upper portion 1104 is designed to fit the upper back of the neck, and lower portion 1106 is designed to fit the base of the neck/shoulders.

Collar 1114 is designed as a height positioner based on the anatomical marker of the shoulder line and/or base of the neck for defining a height relative to the anatomical marker. Collar 1114 includes lower portion 1106 sized and shaped for resting over shoulders of the subject. Optionally, the height of the attached back positioning element is defined to within about 2, 3, 4, 6, 8 centimeters or other values. Upper portion 1104 is designed for optionally centering the attached back positioning element at the neck. Optionally, the spine serves as a centering anatomical marker. Optionally, Upper portion 1104 is designed to at least partially surround the neck on two sides to a degree sufficient to center the back positioning element, for example, in about a 40-60, or about a 80-100 degrees, or about a 150-210 degrees, or about a 215-300 degrees, or about a 245-270 degrees, or about a 245-315 degrees, or over 40 degree, or over 50 degree, or over 90 degree, or over 120 degree arch, or other values, centered at middle of the back of the neck extending around the circumference of the neck, or other range values. Upper portion 1104 is designed to conform to necks of different sizes, for example, diameters of about 6-18 centimeters. Optionally, upper portion 1104 has a semi-circle design with at least one region along the collar with rigidity of below about 100, or 400, or 1000 kN×mm², or other values.

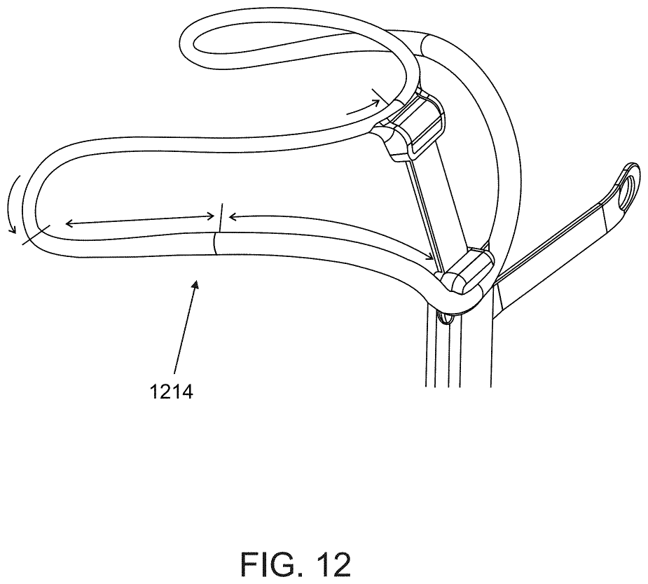

Reference is now made to FIG. 12, which is a schematic of a close up view of a collar 1214 used for height positioning in a back positioning element, in accordance with some embodiments of the present invention. Collar 1214 is connected to the back positioning element, as described herein. Collar 1214 may be implemented as collar 1114 of FIG. 11, as collar 114 of FIG. 1, as collar 214 of FIG. 2 (and as shown in other figures), other implementations as described herein, and/or combinations of the aforementioned.

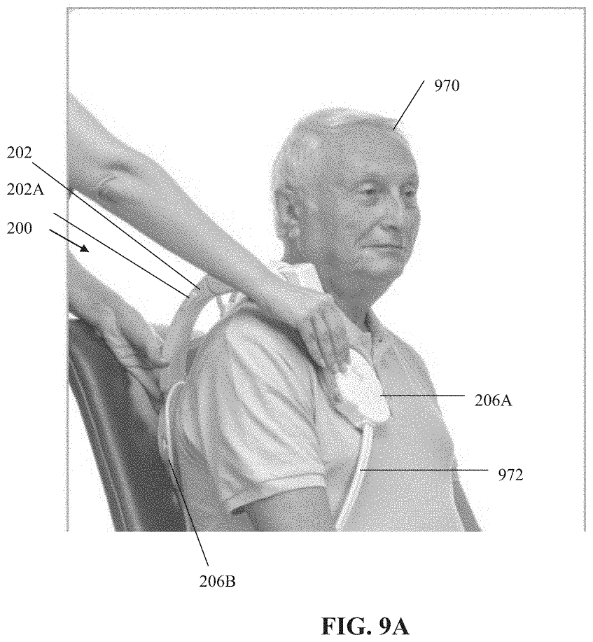

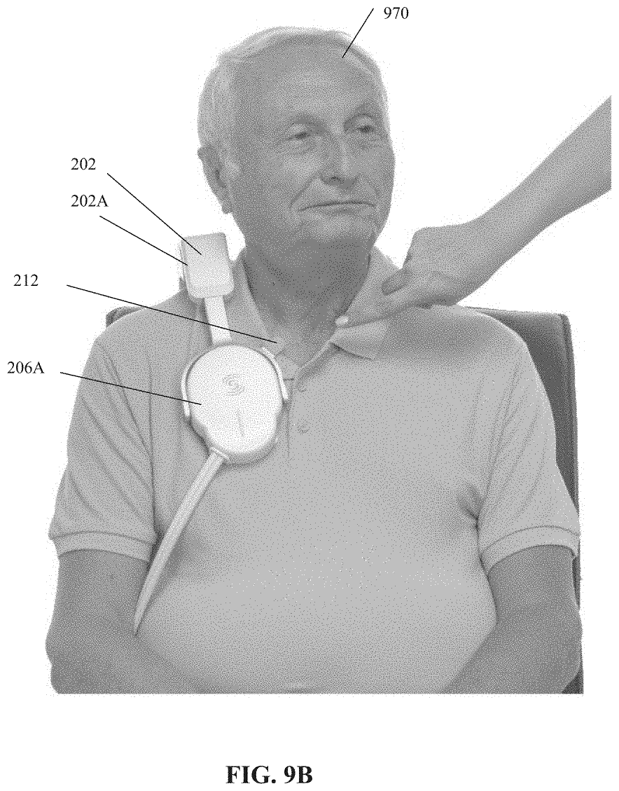

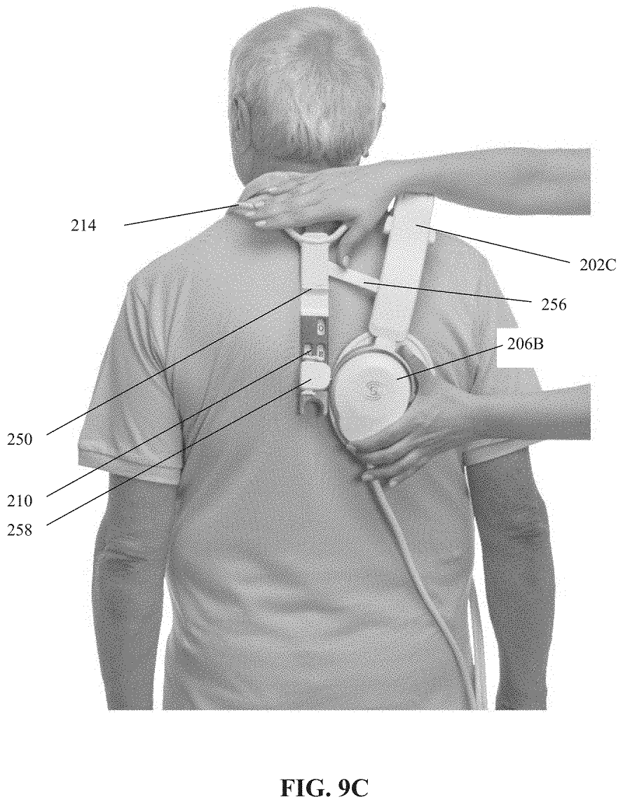

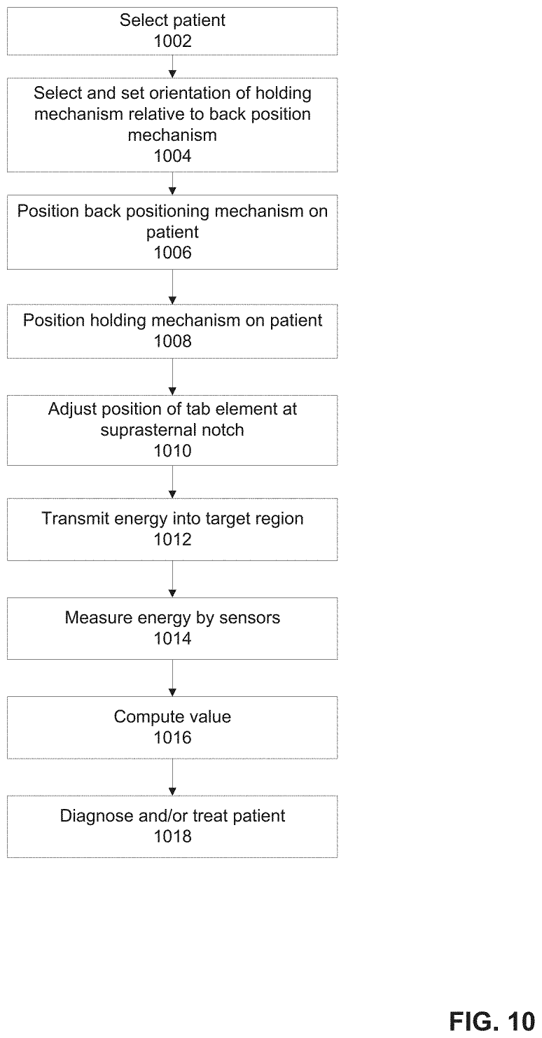

Reference is now made to FIGS. 9A-9C, which are schematics and/or images depicting an example of positioning apparatus 200 on a target individual 970, in accordance with some embodiments of the present invention. Apparatus 200 is as described herein, for example, as described with reference to FIGS. 1-8. Apparatus 200 may be positioned on individual 970 as described herein, for example, as described with reference to FIG. 10. It is noted that cable 972 connects the apparatus with the computing device, as described herein. FIG. 9A is a perspective view of patient 970 wearing apparatus 200. FIG. 9B is a front view. FIG. 9C is a back view.

Reference is now made to FIG. 10, which is a flowchart of a method for positioning the front and/or back sensor(s) across a thorax and at a target region of a target individual, in accordance with some embodiments of the present invention. The method of FIG. 10 may be used with the apparatus described herein, for example, as described with reference to FIGS. 1-9A-C.

At 1002, a patient is selected, for example, treatment by the sensors and/or collecting data by the sensors.

At 1004, one of the possible pre-set positions relative to the holding mechanism relative to the back positioning element is selected optionally using the adjustment mechanism. The selection may be according to the size of the chest.

The selection of setting of the holding mechanism may be based on different data relating to the characteristics of the target individual, for example, anatomical dimensions, weight, height, thorax dimensions, physical condition, age, pathologies, and/or medical conditions, for example conditions affecting the anatomy of the patient. For example, for taller subjects, for example over 155 cm the stop stations to be selected, are, for example, the lower stations 210C or 210D as described with reference to FIG. 6. In another example, for subjects whose chest circumference is bigger than a threshold, for example where the circumferential distance between the sensors is greater than another threshold, the stop stations that may be selected are the rightmost ones, stations 210B or 210D. The stop station may be selected automatically by code, based on an automatic (for example from angular sensors at the hinges of the holding mechanism) or manual input of the characteristic(s) of the target individual.

In another implementation, the holding mechanism may be a fixed sized (e.g., disposable) where different sizes of the holding elements are available, and may be selected for each target individual, based on data relating to the characteristics of the target individual, for example, anatomical dimensions, weight, height, thorax dimensions, physical condition, age, pathologies, and/or medical conditions, for example conditions affecting the anatomy of the patient.

At 1006, the back positioning element is positioned on the back of the patient.

Optionally, the location marker at the bottom region of the elongated element of the back positioning element is positioned at a target location on the spine. When the location marker is in position, the elongated element is located in parallel to and over a long axis of a spine of the target individual. Alternatively or additionally, the collar of the back positioning element is positioned at the base of the back of the neck and/or shoulder line of the target individual.

At 1008, the holding mechanism is positioned over the thorax of the patient. The holding mechanism may be positioned on the left or right side of the patient.

The arc shaped portion of the holding mechanism is positioned over the left or right shoulder of the target individual such that front sensor(s) and back sensor(s) are positioned across the thorax.

In one example, the apparatus is set for positioning the back sensor between about 2-9 centimeters (cm) medially relative to a long axis of the spine, and between about 8-30 centimeters below an upper end of a vertebra prominence of the target patient, for example, when the sensors are EM transducers and about 30% of the effective EM capture and/or transmission area of the EM transducers is in the set target area.

It is noted that 1006 and 1008 may be performed substantially simultaneously and/or iteratively and/or sequentially, for example, 1006 is performed first followed by 1008 by connecting the holding mechanism to the connectors of the back positioning element.

At 1010, the location of the tab element of the holding mechanism is adjusted to the suprasternal notch, placing the front sensor(s) at the correct anatomical location.

At 1012, energy (e.g., RF, EM, ultrasound) may be transmitted from the front and/or back sensor to the target region of the target individual, for example, the lung, a lobe of the lung, an anatomical region of the lung, the heart, trachea, and/or other locations. The energy may be transmitted for diagnosis and/or for treatment (e.g., ablation).

At 1014, the front and/or back sensors measure the energy transmitted through the target region and/or reflected off the target region.

At 1016, one or more values are computed based on the output of the sensors, for example, an amount of fluid in the target region (e.g., lungs, base of the lungs), and/or instructions for adjustment of the applied treatment energy.

At 1018, the target individual (i.e., patient) may be diagnosed and/or treated based on the computed values, for example, treated to remove the excess fluid in the lungs, and/or treated by ablation energy.

It is expected that during the life of a patent maturing from this application many relevant sensors will be developed and the scope of the term sensor is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this present invention may be presented in a range format. It should be understood that the description in range format is merely for convenience and brevity and should not be construed as an inflexible limitation on the scope of the present invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the present invention, which are, for clarity, described in the context of separate embodiments, may also be provided in combination in a single embodiment. Conversely, various features of the present invention, which are, for brevity, described in the context of a single embodiment, may also be provided separately or in any suitable subcombination or as suitable in any other described embodiment of the present invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An apparatus for positioning at least one of a front sensor a back sensor at a target region across a thorax of a target individual, the apparatus comprising:
a back positioning element comprising:
(i) a collar sized and shaped for fitting to a shoulder line and base of a back of a neck of the target individual;
(ii) an elongated element having a first end region connected to the collar, and a second end region with a location marker set to correspond to a target anatomical feature of the spine of the target individual,
wherein when in use, the elongated element is positioned parallel to and over a long axis of a spine on the back of the target individual, and at least one front sensor and at least one back sensor are positioned on the thorax of the patient relative to the back positioning element for transmitting to and/or sensing from the target region.

2. The apparatus of claim 1, further comprising a holding mechanism designed for coupling to the back positioning mechanism, the holding mechanism including:
(i) a substantially arc shaped portion for fitting over a shoulder of the target patient,
(ii) an elongated front portion for connecting to at least one front sensor for contacting a chest of the target individual, and
(iii) an elongated back portion for connecting to at least one back sensor for contacting a back of the target individual,
wherein when in use, the location of the holding mechanism is set relative to the positioned back positioning element, and the at least one front sensor and the at least one back sensor are positioned on the thorax of the patient by the holding mechanism relative to the back positioning mechanism for transmitting to and/or sensing from the target region.

3. The apparatus of claim 2, further comprising: a non-transitory memory having stored thereon a code that when executed by at least one hardware processor of a computing device causes the at least one hardware processor to: control activation of the at least one back sensor and the at least one front sensor, receive output of the at least one back sensor and the at least one front sensor, and compute an estimate of an amount of fluid within the target tissue of the target individual according to the output of the at least one back sensor and the at least one front sensor.

4. The apparatus of claim 1, wherein the back positioning element has an approximately Y shape, the collar having a shape approximately as the short top arms of the Y, and the elongated element of the back positioning element having a shape approximately as the long arm of the Y.

5. The apparatus of claim 2, wherein the holding mechanism is shaped as U having a first arm shorter than a second arm, the front portion of the holding mechanism corresponding to the first arm of the U, the back portion of the holding mechanism corresponding to the second arm, and the arc shaped portion of the holding mechanism corresponding to the curve of the U.

6. The apparatus of claim 2, wherein the back positioning element further comprise an adjustment mechanism for adjusting a distance between the holding mechanism and the elongated element of the back positioning element, wherein the adjustment mechanism includes a plurality of stop station selectors arranged in a two dimensional pattern, each stop station selector is set at a predefined location of the holding mechanism for a setting a different distance of the holding mechanism relative to the elongated element of the back positioning element.

7. (canceled)

8. The apparatus of claim 6, wherein each one of the plurality of stop station selectors corresponds to a combination of at least one physical and/or anatomical patient parameters.

9. The apparatus of claim 6, wherein the adjustment mechanism adjusts the location of the holding mechanism within a two dimensional plane substantially parallel to the back of the target individual.

10. The apparatus of claim 2, wherein front portion element includes at least one tab element set such that in use when the at least one tab element is positioned at a defined anatomical landmark of the target individual, and the at least one front sensor is positioned relative to the chest at a location corresponding to the target region.

11. (canceled)

12. The apparatus of claim 2, wherein the holding mechanism, and the back positioning element are sized, shaped, and set for positioning the at least one front sensor and the at least one back sensor across the target region located within at least one member selected from the group consisting of: left lung, right lung, right middle lobe, right upper lobe, right lower lobe, left upper lobe, right lower lobe, heart, trachea, and combinations of the aforementioned.

13. The apparatus of claim 2, further comprising at least one front sensor positioning element coupled to the at least one front sensor for adjustment of an orientation of the at least one front sensor for contacting the chest of the target individual corresponding to the target region, the at least one front sensor positioning element coupled to the front portion of the holding mechanism, and/or at least one back sensor positioning element coupled to the at least one back sensor for adjustment of an orientation of the at least one back sensor for contacting a back of the target individual corresponding to the target region, the at least one back sensor positioning element coupled to the back portion of the holding mechanism.

14. The apparatus of claim 2, wherein the holding mechanism includes a plurality of rigid sections connected by spring loaded hinges designed for adjustment of a distance between the at least one front sensor and the at least one back sensor and for application of a spring force for urging the at least one back sensor and/or the at least one front sensor to the thorax of the target individual.

15. The apparatus of claim 1, wherein the collar is sized and/or shaped to cover an arch of about 40-60 degrees of the back of the neck of the target individual.

16. The apparatus of claim 1, wherein the sensors are selected from the group consisting of: electromagnetic (EM) transmitters and/or receivers, ultrasound transmitters and/or receivers, radiofrequency (RF) transmitters and/or receivers, treatment elements, chemical injectors, and imaging elements.

17. The apparatus of claim 2, wherein the holding mechanism made up of an elastic material set for increasing an distance between the at least one front sensor and the at least one back sensor for accommodating the thorax and for urging the at least one front sensor and the at least one back sensor towards one another, wherein when in use, the holding mechanism applies a force to the at least one front sensor and the at least one back sensor against the thorax at the position corresponding to the target region.

18. The apparatus of claim 1, wherein the apparatus is set for positioning the at least one back sensor between about 2-9 centimeters (cm) medially relative to a long axis of the spine, and between about 8-30 centimeters below an upper end of a vertebra prominence of the target patient.

19. (canceled)

20. The apparatus of claim 1, further comprising additional sensors for computing a distance between the front sensor and back sensor when the apparatus is in use.

21. A method for positioning at least one front and at least one back sensor at a target region across a chest of a target individual, the method comprising:
providing the apparatus of claim 2;
positioning the collar of the back positioning element on the shoulder line and/or base of the neck of the target individual;
positioning the location marker of the elongated element of the back positioning element at a predefined anatomical landmark s of a spine of the target individual;
positioning the arch shaped portion of the holding mechanism over a shoulder of the target individual such that at least one front sensor and at least one back sensor coupled to the holding mechanism are positioned across the thorax; and
adjusting a position of a tab element coupled to the elongated front portion of the holding mechanism at a predefined anatomical location on the chest.

22. The method of claim 21, further comprising transmitting EM energy between the at least one front sensor and the at least one back sensor across the chest and through the target region of the target individual; measuring the EM energy transmitted through the target region; and computing an amount of fluid in the target region.

23. The method of claim 21, further comprising selecting one of a plurality of stop stations of the holding mechanism relative to the elongated element of the back positioning element according to one of the plurality of stop stations corresponding to a combination of at least one anatomical and/or physical dimensions of the thorax of the target individual.

24. (canceled)
